(19) United States
(12) Patent Application Publication
(10) Pub. No.: US 2018/0092852 A1
(43) Pub. Date: Apr. 5, 2018

(54) Novel Method for Producing Hollow Shells from Pollen Grains

(71) Applicant: Texas Tech University System, Lubbock, TX (US)

(72) Inventors: Harvinder Singh Gill, Lubbock, TX (US); Shashwati U. Atwe, Lubbock, TX (US); Pedro E. Gonzalez-Cruz, Lubbock, TX (US)

(21) Appl. No.: 15/725,099

(22) Filed: Oct. 4, 2017

Related U.S. Application Data
(60) Provisional application No. 62/404,005, filed on Oct. 4, 2016.

(51) Int. Cl. A61K 9/48; A61K 9/50

(52) U.S. Cl. CPC A61K 9/4816; A61K 36/00; A61K 9/5068; A61K 9/4858

(57) ABSTRACT

The present invention includes methods of making a hollow exine shell from pollen grains comprising the steps of: providing a plant pollen or spore; extracting organic matter from the plant pollen or spore with an organic solvent; after the organic extraction treating the plant pollen or spore with an acid solution; after the acid treatment treating the plant pollen or spore with an alkali solution; and isolating the plant pollen or spore, wherein the pollen or spore have open apertures on pollens with visible apertures that open to the interior hollow cavity, wherein the same apertures are closed in naturally occurring pollens.

### CROSS-REFERENCE TO RELATED APPLICATIONS

This application claims priority to U.S. Provisional Application Ser. No. 62/404,005, filed Oct. 4, 2016, the entire contents of which are incorporated herein by reference.

### STATEMENT OF FEDERALLY FUNDED RESEARCH

This invention was made with government support under 1DP2HD075691-01 awarded by National Institutes of Health. The government has certain rights in the invention.

### TECHNICAL FIELD OF THE INVENTION

The present invention relates generally to methods, compositions and formulations for producing hollow exine shells from pollen grains.

### INCORPORATION-BY-REFERENCE OF MATERIALS FILED ON COMPACT DISC

None.

### BACKGROUND OF THE INVENTION

Without limiting the scope of the invention, its background is described in connection with vaccinations. Vaccinations are an effective and cost-efficient means of protecting against infectious agents; however, injecting vaccines using a hypodermic needle is not the most convenient, likable, or safe method of vaccination. The use of hypodermic needles results in significant pain and discomfort to patients, requires trained personnel for administration, and can cause accidental needle-pricks resulting in transmission of blood borne pathogens such as HIV and hepatitis virus. In contrast, oral administration of vaccination is painless, is the most convenient to use, and can result in high patient compliance. It also has the potential to allow self-administration of vaccines and can allow rapid distribution of vaccines to the public in case of pandemics. Furthermore, processing of locally delivered antigens in the gut-associated lymphoid tissues (GALT) can induce strong mucosal immunity in the gut and other distant mucosal surfaces. On the other hand, the systemic delivery of vaccines using hypodermic needles is a poor stimulator of mucosal immunity. Mucosal immunity is important because mucosal surfaces such as the gut-lining and the respiratory epithelium form a major portal of entry for pathogens, and neutralization of pathogens on mucosal surfaces can form a first line of defense. Thus, overall the oral route of a vaccination is not only safer, convenient and painless, but it is also expected to be functionally superior due to the potential of stimulating both the systemic and the mucosal arms of immunity.

Pollen grains have served as delivery vehicles for their naturally-contained genetic material and proteins for pollination and are natural delivery devices for macromolecules the size of proteins and nucleic acids, as well as for smaller molecules. They are also useful as delivery systems outside of their natural function in pollination. Their surfaces adhere to tissue surfaces and particularly to mucous membranes and remain in contact for prolonged periods of time to release the substances contained therein to the blood stream or circulatory system. For example, U.S. Pat. No. 7,608,270, entitled, “Dosage Form,” discloses a pharmaceutical or dietetic dosage form comprising of effective quantity of an active substance chemically or physically bound to support comprising sporopollenin, or other similar exine coating of spores, of a plant or fungus, optionally with further excipients.

For example, U.S. Pat. No. 7,846,654, entitled, “Uses of Sporopollenin” discloses the use of an exine shell of a naturally occurring spore, or a fragment thereof, as an antioxidant, for instance in a composition or formulation containing an active substance. Also provided is a method for reducing rancidity, or other oxidative degradation, of a substance, composition, or formulation, by encapsulating the substance, composition, or formulation in, or chemically binding it to, or mixing it with, an exine shell of a naturally occurring spore or a fragment thereof. These patents achieved significant removal of plant native proteins not seen in earlier studies and specify that the pollen grain shell must have protein content less than 0.5% of the exine coating. Based on this qualification the inventors of patent ‘a’ and ‘b’ were able to have new patents issued.

For example, U.S. Pat. No. 5,013,552, entitled, “Modified Pollen Grains for Delivering Biologically Active Substances to Plants and Animals,” discloses loaded pollen grains which are suitable for use as delivery systems for introducing biologically active substances into or on plants and animals. Such pollen grains are suitable to deliver both small and large (macromolecules) molecules. Preferred pollen grains are those that have been defatted and then pre-treated to be free of antigenic materials and that have special surface features that facilitate their attachment to tissue surfaces, particularly to mucous membranes. The most preferred pollen grains are those that have spiny or irregular or fragmented surfaces. Also disclosed are a method of pre-treating the pollen grains to remove antigenic materials; a method of loading the pollen grains with the biologically active material; and a method of incorporating such pre-treated, loaded pollen grains into formulations or dosage forms suitable for introduction into or on a plant or animal body.

For example, U.S. Pat. No. 5,275,819, entitled, “Drug loaded pollen grains with an outer coating for pulsed delivery,” discloses a pulsating release composition comprising natural microspheres, such as pollen grains or spores, into which are loaded a biologically active that is subsequently releasable therefrom in a predetermined location in or on a plant or animal in a series (generally 3 or more) of pulses. The composition comprises a group of substantially similar loaded microspheres coated with multiple barrier layers alternating with multiple active substance layers in a concentric onion-like structure, the barrier layers being slowly soluble to delay release of active substance from the underlying layer thereof until after the pulse of active substance provided by the overlying layer has subsided. In another preferred embodiment, the composition comprises a plurality of loaded microspheres divided into as many fractions as the desired number of pulses, the loaded microspheres in each consecutive fraction being coated with a barrier layer adapted to dissolve consecutively more slowly to delay release of active substance from such fraction until after the pulse of active substance provided by the prior fraction of consecutively more soluble barrier-coated microspheres has subsided. In another aspect of the invention, the active substance-containing bodies in the compositions may be coated with one or a mixture of absorption-promoting enzymes.

### SUMMARY OF THE INVENTION

In one embodiment, the present invention includes a method of making a hollow exine shell from pollen grains comprising the steps of: providing a plant pollen or spore; extracting organic matter from the plant pollen or spore with an organic solvent; after the organic extraction treating the plant pollen or spore with an acid solution; after the acid treatment treating the plant pollen or spore with an alkali solution; and isolating the plant pollen or spore, wherein the pollen or spore have open apertures on pollens with visible apertures that open to the interior hollow cavity, wherein the same apertures are closed in naturally occurring pollens. In one aspect, the method further comprises the step of changing the times for at least one of the organic extraction, acid treatment, or the alkali treatment to optimize the size of the apertures. In another aspect, the method further comprises the step of changing the strength of the acid to optimize the size of the aperture of the plant pollen or spore. In another aspect, the method further comprises the step of changing the strength of the alkali to optimize the size of the aperture of the plant pollen or spore. In another aspect, the method further comprises the step of adding an antigen selected from bacteria, viruses, fungi, protozoans, parasites, prions, toxins, cancer, or allergens to modulate an immune response to the antigen. In another aspect, the method further comprises the step of adding one or more antigens comprising oligonucleotides, proteins, peptides, deoxyribonucleic acid (DNA), ribonucleic acid (RNA), cells (broken or intact), lipids, toxin variants, carbohydrates, virus-like particles, liposomes, live attenuated or killed natural or recombinant microorganisms, bacteria, viruses, and particulate vaccine delivery systems, liposomes, virosomes, polymeric/inorganic/organic micro and nanoparticles, immune stimulating complexes (ISCOMS) and combinations thereof, wherein antigens are in composition or can be attached/adsorbed/anchored physically or chemically to pollen/spore at the exterior surface, interior surface/cavity or pores. In another aspect, the plant pollen or spore is formed into a vaccine composition that is adapted for oral, nasal, pulmonary, rectal, optical, transdermal, transmucosal, intramuscular, or subcutaneous delivery. In another aspect, the method further comprises adding a cryoprotectant selected from trehalose or other sugars/carbohydrates. In another aspect, the method further comprises the step of coating the treated plant pollen or spore with a coating. In another aspect, the method further comprises the step of adding an adjuvant to the treated plant pollen or spore. In another aspect, the method further comprises the step of adding a polymer coating applied to the pollen/spore, wherein the polymer coating is a diffusion barrier, a coating that includes physical or chemical adsorption/attachment/anchoring points, plugs one or more of the multiple pores, coats the inner cavity, coats the exterior surface or a combination thereof. In another aspect, the method further comprises the step of adding at least one of an adjuvant or an antigenic protein to the treated plant pollen or spore. In another aspect, the isolated pollen is at least one of: substantially free of proteins, substantially free to antigenic proteins, free of proteins, or free of antigenic proteins. In another aspect, each steps of extracting, or treating are followed by a vacuum filtration and washing step.

In yet another embodiment, the present invention includes an open pore plant pollen or spore made by a method that comprises the steps of: providing a plant pollen or spore; extracting organic matter from the plant pollen or spore with an organic solvent; after the organic extraction treating the plant pollen or spore with a hot strong acid solution; after the acid treatment treating the plant pollen or spore with a hot strong alkali solution; and isolating the plant pollen or spore, wherein the pollen or spore have open apertures on pollens with visible apertures that open to the interior hollow cavity, wherein the same apertures are closed in naturally occurring pollens. In one aspect, the method further comprises the step of changing the times for at least one of the organic extraction, acid treatment, or the alkali treatment to optimize the size of the apertures. In another aspect, the method further comprises the step of changing the strength of the acid to optimize the size of the aperture of the plant pollen or spore. In another aspect, the method further comprises the step of changing the strength of the alkali to optimize the size of the aperture of the plant pollen or spore. In another aspect, the method further comprises the step of adding an antigen selected from bacteria, viruses, fungi, protozoans, parasites, prions, toxins, cancer, or allergens to modulate an immune response to the antigen. In another aspect, the method further comprises the step of adding one or more antigens comprising oligonucleotides, proteins, peptides, deoxyribonucleic acid (DNA), ribonucleic acid (RNA), cells (broken or intact), lipids, toxin variants, carbohydrates, virus-like particles, liposomes, live attenuated or killed natural or recombinant microorganisms, bacteria, viruses, and particulate vaccine delivery systems, liposomes, virosomes, polymeric/inorganic/organic micro and nanoparticles, immune stimulating complexes (ISCOMS) and combinations thereof, wherein antigens are in composition or can be attached/adsorbed/anchored physically or chemically to pollen/spore at the exterior surface, interior surface/cavity or pores. In another aspect, the plant pollen or spore is formed into a vaccine composition that is adapted for oral, nasal, pulmonary, rectal, optical, transdermal, transmucosal, intramuscular, or subcutaneous delivery. In another aspect, the method further comprises adding a cryoprotectant selected from trehalose or other sugars/carbohydrates. In another aspect, the method further comprises the step of coating the treated plant pollen or spore with a coating. In another aspect, the method further comprises the step of adding an adjuvant to the treated plant pollen or spore. In another aspect, the method further comprises the step of adding a polymer coating applied to the pollen/spore, wherein the polymer coating is a diffusion barrier, a coating that includes physical or chemical adsorption/attachment/anchoring points, plugs one or more of the multiple pores, coats the inner cavity, coats the exterior surface or a combination thereof. In another aspect, the isolated pollen is at least one of: substantially free of proteins, substantially free to antigenic proteins, free of proteins, or free of antigenic proteins. In another aspect, each steps of extracting, or treating are followed by a vacuum filtration and washing step.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic representation of the conventional and new treatment method of the present invention.

FIGS. 2A to 2D show comparative figures that demonstrate the effect of the novel treatment of the present invention on Lycopodium spores and Ragweed pollens. FIG. 2A shows a conventional treatment when used with Lycopodium spores gives intact pollen shells with a clean interior, FIG. 2B shows the conventional treatment for Ragweed pollens results in irrecoverable pollens, FIG. 2C shows the (switched) treatment method when used for Lycopodium spores causes them to rupture at or near the trilete scar and FIG. 2D shows the new (switched) treatment method when used for Ragweed pollens gives intact pollen shells with a clean interior.

FIGS. 3A to 3D show SEM images of Lycopodium spores (LSs) processed using the conventional treatment (CT). Raw LS: FIG. 3A shows the exterior showing the original morphology and FIG. 3B shows the interior showing the presence of natural biological material. LS processed using CT: FIG. 3C shows the exterior showing an intact morphology and FIG. 3D shows a clean interior made using the present invention.

FIGS. 4A to 4G show a schematic diagram and images of LSs and RW pollens processed using the CCT and results therefrom. FIG. 4A is a schematic diagram of the processing steps on the CCT protocol. FIG. 4B shows LSs pollens after processing with CCT and FIG. 4C is a zoomed-in image of a single pollen. RW pollens after 6 hours of KOH treatment: FIG. 4D is a photograph of the flake formed after vacuum filtration. FIG. 4E is an SEM image of the flake showing pollen entrapped in extraneous materials. FIG. 4F is a zoomed-in SEM image of the flake showing more details of entrapped pollens.

FIGS. 5A to 5I show images of RW pollens processed using the CCT and MCCT after 12 hours of KOH and MCCT after 7 days of phosphoric acid treatment. FIG. 5A shows a photograph of the flake formed after vacuum filtration. FIG. 5B shows an SEM image of the flake showing pollen entrapped in extraneous materials. FIG. 5C shows a zoomed in SEM image of the flake showing more details of entrapped pollens. FIG. 5D is a schematic diagram of the processing steps for figures FIG. 5A to FIG. 5B (vacuum filtration) and FIG. 5E to FIG. 5F (centrifugation). FIG. 5E. Clumps formed after centrifugation and FIG. 5F is a zoomed-in SEM image of the clumps showing more details of entrapped pollens. FIG. 5G is a schematic diagram of the processing steps for figures FIG. 5H and FIG. 5I. FIG. 5H is an SEM image of pollens clumped together and entrapped due to extraneous materials. FIG. 5I is a zoomed-in SEM image of the clump showing more details of entrapped pollens with unclean surfaces.

FIGS. 6A to 6J show SEM images of Ragweed (RW) pollens processed using the switched treatment (SCT). FIG. 6A shows a comparison diagram of the CCT and SCT treatment steps. Raw RW pollens: FIG. 6B is a zoomed-out image of multiple raw RW pollens, FIG. 6C shows an image of the exterior of the pollen showing the original morphology and FIG. 6D is a image that shows the interior of the pollen showing the presence of natural biological materials. RW pollens processed at high temperatures (SCTH): FIG. 6E is a zoomed-out image of multiple pollens after SCTH, FIG. 6F is an image of the exterior of a pollen showing an intact morphology and FIG. 6G is an image showing a clean interior of the processed pollen. RW pollens were processed at low temperatures (SCTL): FIG. 6H is a zoomed-out image of multiple pollen after SCTL, FIG. 6I is an image of the exterior of the pollen showing an intact morphology and FIG. 6J is am image showing a clean interior of the pollen.

FIG. 7 is a graph that shows protein content of hollow exine shells obtained using the switched protocol A. The percent protein content of raw pollens and the ones processed by SCTH and SCTL show a considerable reduction indicating success of the process in removal of native proteinaceous material.

FIG. 8 shows a Fourier-transform infrared spectroscopy (FTIR) spectra of SCT processed RW pollen. Natural ragweed pollens were treated with acetone, phosphoric acid, and potassium hydroxide sequentially at two different temperatures. Low-temperature method used phosphoric acid and potassium hydroxide treatment at 60° C. and 80° C., respectively while high-temperature method used phosphoric acid and potassium hydroxide treatment at 160° C. and 120° C., respectively.

FIGS. 9A to 9H show SEM images of other species of pollens processed using the SCTL protocol. FIG. 9A is a zoomed-out image of Chenopodium album (Lambs quarter (LQ)), FIG. 9B is an image that shows intact processed pollen grain and FIG. 9C shows the clean interior achieved with the SCTL protocol. FIG. 9D is a zoomed-out image of Helianthus annus (Sunflower) pollens, FIG. 9E shows an intact processed pollen grain and FIG. 9F show a clean interior achieved using the SCTL protocol of the present invention. FIG. 9G is a zoomed-out image of Lycopodium clavatum pollens and FIG. 9H shows broken processed pollen grain as a result of the SCTL protocol.

FIGS. 10A and 10B show images of pollen apertures bursting due to osmotic pressure buildup. FIG. 10A shows Lambs Quarter (LQ) pollens before and after exposure to ortho-phosphoric acid showing the buildup of pressure that will cause the opening to burst open to release it. FIG. 10B shows LQ pollens SEM images after exposure to other solvents that did not cause a buildup in pressure.

FIG. 11 shows a proposed mechanism of pore opening in pollen grains. At point A, a diagram of a pollen grain with its different components is shown. In pathway B, a diagram of pollen without aperture exposed to an environment that causes a build up in osmotic pressure, which release will be at a weak spot on the pollen wall. In pathway C, a diagram of pollen with an aperture where the buildup pressure will be release through the pores.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In the description of this patent ‘conventional chemical treatment (CCT)’ is used interchangeably with ‘conventional treatment (CT)’. The terminology ‘new (switched) treatment’ is used interchangeably with ‘switched chemical treatment (SCT)’.

Pollens/spores are hollow shells that contain plant reproductive material. Their outer wall is made of a very tough biopolymer called sporopollenin that protects the reproductive material from various physical, chemical and environmental assaults. Sporopollenin can also withstand the acidic environment of the stomach. Surprisingly, despite their relatively large size (˜30 μm in diameter) it has been found that pollens/spores can travel as intact particles across the intestine into the blood in humans and animals. Furthermore, pollen/spore shells are naturally porous to allow exchange of gases, water and nutrients required by the plant reproductive structure residing inside. The present inventors have realized that these properties of pollens/spores suggest a unique opportunity to exploit pollens/spores for oral (and via other routes and approaches) drug transport because pores in the pollen/spore shell could be used to first extract the native material from inside the pollens/spores, and then could be used to again fill the clean interior space with drug molecules, the chemically resistant shell of pollens/spores could safely transport drugs loaded in its interior across the harsh environment of the stomach, and upon reaching the intestines, the drug-filled pollens/spores could move into the human body carrying the drug with them. This conceptual framework has been reduced to practice. Pollens/spores can be readily cleaned with inexpensive chemicals, and then filled with molecules using mild vacuum that does not expose biological or chemical drugs to harsh denaturing conditions. It has been shown that proteins as large as 540 kDa, and a magnetic resonance imaging contrast agent, food oils including cod liver oil can be filled into pollens/spores.

Pollens/spores are part of traditional medicine across the world including India, China, American Indians, Turkish folk medicine, and Papua New Guinea to name a few. They are used to treat a number of ailments including kidney disorders and stomachache. From a more scientific western-research perspective two studies exist which show that feeding untreated or treated lycopodium spores to humans does not cause any adverse effects. First is a study done in 1974 where untreated lycopodium spores were fed to human subjects to study kinetics of lycopodium spore absorption into blood, and the second is a study where chemically-treated lycopodium spores were mixed with fish oil and fed to humans to help mask the foul taste of fish oil. Together, these different observations provide confidence that both native and cleaned lycopodium spores are safe for human oral consumption.

Organic solvents for use in the organic solvent extraction of the present invention include, e.g., acetone, acetate, acetaldehyde, acetamide, acetonitrile, 1-butanol, 2-butanol, sec-butanol, t-butanol, dihydropyran, 3-methyl-1-butanol, 2-methyl-1-butanol, 3-methyl-2-butanol, 2-methyl-2-butanol, ethanol, ethyleneglycol, ethyleneglycol monomethyl ether, diethyl ether, methylethyl ether, ethylpropyl ether, ethyl propionate, ethyl acetate, ethylmethyl ketone, furan, isopropanol, methanol, methylpropyl ether, 1,2-dimethoxyethane, tetrahydrofuran, dihydrofuran, 1-pentanol, 2-pentanol, 3-pentanol, neopentanol, propanol, pyran, tetrahydropyran, methyl acetate, propyl methylformate, ethylformate, methyl propionate, dichloromethane, chloroform, dimethylformamide, dimethylacetamide, N-methylpyrrolidone, diethyl ketone, propionitrile, or combinations thereof.

The composition made using the present invention may be adapted for administration via, e.g., oral, topical, parenteral, intramuscular, subcutaneous, intradermal, vaginal, rectal, intracranial, intranasal, intraocular, auricular, pulmonary intralesional, intraperitoneal, intraarterial, intracerebral, intracerebroventricular, intraosseus and intrathecal administration.

A study done in humans in 1974 demonstrated that after oral ingestion of lycopodium clavatum spores, 6,000 to 10,000 spores per human volunteer were absorbed into the blood stream where they could be detected by electron microscopy. This clearly shows that lycopodium spores can enter the human body across the intestinal mucosa as intact particles. It was further observed in the study that lycopodium spores in the blood defragmented (perhaps due to enzymatic action) and were cleared from the body, providing a natural mechanism of lycopodium spore clearance.

There is nothing in the art related to pollens/spores that mentions, suggests or implies any immunological potential of pollen/spores. These patents/publications only teach that therapeutic agents, food additives and nutraceuticals can be delivered using pollens/spores. There is nothing in the art that provides that clean pollens/spores that are substantially cleaned to remove native pollen proteins may have potential for vaccination and that pollens/spores may boost immune response to vaccines/antigens.

The composition may be for suitable and/or adapted and/or intended for topical delivery of an active substance to a surface, in which case the surface may be a living surface (again, either plant or animal) or an inanimate surface. The ability of the pollen/spore to act as a physical barrier protecting an encapsulated active substance, can be of particular significance in this context, since on release of the active substance onto a surface, the substance will then be exposed on the outside of the pollen/spore.

Several chemical^{[1-6]} and enzymatic methods^{[7, 8]} exist that can be used to obtain hollow pollen exine shells. The conventional methods involve the use of organic solvents for defatting pollens, followed by alkali treatment to remove the proteinaceous material from within pollens and then treatment with an acid as the last step to remove the intine.^{[9-11]} This method, though successful for obtaining SECs from Lycopodium clavatum spores, is known to fail for other more delicate species of pollens.^{[12]} This has made obtaining intact pollen shells of species other than Lycopodium clavatum a challenge. Hence, there is need to develop an optimized and robust treatment procedure that can produce intact SECs with low protein content consistently and that can be applied to pollens from different sources.

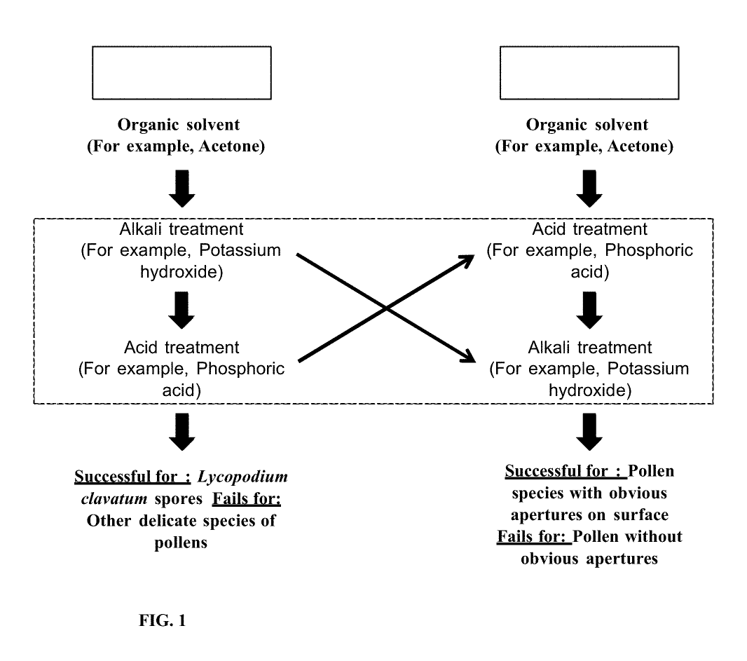

In this invention, a new method for the treatment of such species of pollens is disclosed. The method involves defatting pollens with an organic solvent, followed by treatment with hot acid and finally treating them with hot alkali. The results show that this method can be successfully applied to multiple species of pollens. FIG. 1 is a schematic representation of the conventional and new treatment method of the present invention. However, this method was found to induce considerable damage to Lycopodium clavatum spores with the spores rupturing open during the treatment. Based on this observation it can be said this process can be successfully applied only to those species of pollens, which have obvious apertures on their surface. By way of explanation, and in no way a limitation of the present invention, the new treatment method induces osmotic stress (physical/chemical/mechanical/otherwise) which may be temperature induced/pressure induced/concentration induced. This osmotic stress build up inside the pollen can cause them to burst open at the pores and release the inside material to their surroundings. The osmotic bursting of pollens is a common phenomenon in nature.^{[13, 14]} However, if the pollens have no obvious apertures on their surface (for example, Lycopodium clavatum spores) the osmotic stress build up cannot be relieved. This can cause the pollens to burst them open to release the inside material.^{[15]}

Thus, this new invention has been successfully used with only those species of pollens that have visible apertures on their surface. When applied to such species the invention is very successful in producing intact pollen shells with a clean interior and maximum removal of native pollen material. These hollow pollen shells can then be successfully used for several different applications. The importance of this invention becomes clear by noting that a previous attempt by Mundargi et al. (Mundargi R C, Potroz M G, Park J H, Seo J, Lee J H, Cho N J. Extraction of sporopollenin exine capsules from sunflower pollen grains. RSC Advances 2016; 6(20):16533-9.) to clean sunflower pollens did not lead to low protein content. They could only achieve the lowest protein content in pollens at about 4% by mass. Mundargi et al. also note that when they tried alkali treatment, they saw that sunflower pollens were damaged. However, in the protocol described herein by the inventors, they have successfully reduced the protein content of pollens (including sunflower pollen) to about 1% or even lower in certain pollens. The ability to use alkali treatment after acid treatment was one important factor in achieving this reduced protein content.

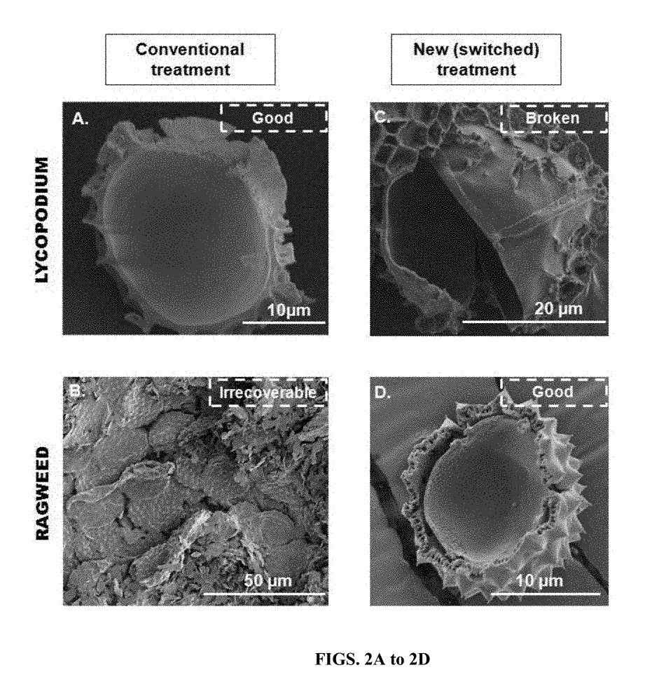

FIGS. 2A to 2D are comparative figures that show the effect of the novel treatment of the present invention on Lycopodium spores and Ragweed pollens. FIG. 2A shows a conventional treatment when used with Lycopodium spores gives intact pollen shells with a clean interior, FIG. 2B shows the conventional treatment for Ragweed pollens results in irrecoverable pollens, FIG. 2C shows the (switched) treatment method when used for Lycopodium spores causes them to rupture at or near the trilete scar and FIG. 2D shows the new (switched) treatment method when used for Ragweed pollens gives intact pollen shells with a clean interior.

Pollen grains or spores are naturally occurring microcapsules produced by plants to transport the male gametes from the anther (male part) to stigma (female part) where fertilization takes place.^{[16-18]} During this transportation, the pollen grain protects the male gametes from environmental stresses due to its unique structure.^{[19]} A typical pollen grain or spore has a tough outer shell known as the exine, an inner shell made up of cellulose and pectin known as the intine and a hollow inner cavity which holds the male gametes and other biomolecules and nutrients.^{[10, 20-22]} The exine is primarily made up of a biopolymer known as sporopollenin, the exact structure of which is unknown.^{[10]} This sporopollenin is known to be very tough and resistant to extreme temperatures, various organic solvents, acids and alkalis.^{[10, 23-26]} Due to this property obtaining hollow exine shells, also known as sporopollenin exine capsules (SECs), is easily possible by subjecting natural pollen grains to a series of chemical treatment steps to remove the intine and inner biomolecules and obtain hollow capsules that can then be loaded with any material of interest. These SECs obtained are becoming increasingly popular for a variety of applications such as drug and vaccine delivery, encapsulation of contrast agents, cells and micro-organisms and even taste masking.^{[10, 27-32]}

Several chemical^{[1-6]} and enzymatic methods^{[7, 8]} exist that can be used to obtain hollow pollen exine shells. The conventional method involves the use of organic solvents for defatting pollens, followed by alkali treatment to remove the proteinaceous material from within pollens and then treatment with an acid as the last step to remove the intine.^{[9-11]} This method, though successful for obtaining SECs from Lycopodium clavatum spores, is known to fail for other more delicate species of pollens.^{[12]} Hence, there is need to develop an optimized and robust treatment procedure that can produce intact SECs with low protein content consistently and that can be applied to pollens from different sources.

The inventors investigated the cause of failure of the conventional treatment method to work for more delicate species of pollens. Ambrosia elatior (Common ragweed) pollen was used for this purpose. To obtain clean and intact SECs with ragweed (RW) pollens, the conventional method and several modifications of it were not found to be successful. In all attempts the RW pollens were found not to survive this treatment. Hence, a new method was developed that involved switching the sequence of the alkali and acid treatment steps. The surprising success of this method was confirmed by elemental and scanning electron microscopic analysis. The results indicate that the new processing method proposed is capable of producing intact SECs with a clean interior of RW pollens. This method was also found to be successful with other species of pollens, except for LSs in which case it was found to induce considerable damage.

Raw Lycopodium clavatum spores were obtained from Sigma Aldrich (MO, USA), raw Ambrosia elatior (Common Ragweed) was purchased from Pharmallerga (Li\check{s}ov, Czech Republic). Acetone, potassium hydroxide, orthophosphoric acid, ethanol, hydrochloric acid and sodium hydroxide were purchased from Fisher Scientific (PA, USA), centrifuge tubes were purchased from Corning (NY, USA), Milli-Q water (Millipore, Mass., USA) with a resistance of 18.2 MΩcm was used in all experiments was used for all experiments.

Different treatment schemes were used in this study. All of them were used for Lycopodium spores (LSs) and ragweed pollens (RW). These treatments are described in detail as follows.

Conventional treatment (CT): 50 g of pollens were stirred in 450 ml of acetone under reflux overnight at 65° C. These were then air dried overnight and transferred to 600 ml of 6% potassium hydroxide solution (KOH). This solution was refluxed at 120° C. for 12 hours with the solution renewed at 6 hours. These alkalis treated pollens were then filtered, washed with hot water (3×300 ml) and hot ethanol (3×300 ml) and air dried overnight. Then these spores/pollens were stirred under reflux in 900 ml of orthophosphoric acid for 7 days at 160° C. On the 8^{th} day the pollens were filtered and washed with water (5×300 ml), acetone (300 ml), 2 mol/L hydrochloric acid (300 ml), 2 mol/L sodium hydroxide (300 ml), water (5×300 ml) and ethanol (2×300 ml). Following these washings the treated pollens were dried at 60° C. in a hot air oven until constant weight was achieved.

Modified conventional treatment (MCT): 50 g of pollens were stirred in acetone (450 ml) under reflux overnight at 65° C. These were then air dried overnight and transferred to 600 ml of 6% potassium hydroxide (KOH) solution. This solution was stirred under reflux for 6 hours, cooled to room temperature and centrifuged. The supernatant KOH was discarded and fresh KOH solution (50 ml) was added to the tube. This solution was transferred to a round bottom flask containing remaining KOH (550 ml) and stirred under reflux as before for 6 hours. After 6 hours the centrifugation step was repeated, KOH discarded and the pollens were transferred to orthophosphoric acid (900 ml) and refluxed at 160° C. for 7 days. The recovered pollens were washed as mentioned in CT and dried at 60° C. in a hot air oven till constant weight was achieved.

Switched treatment (ST): 20 g of pollens were stirred under reflux in acetone at 65° C. overnight. After reflux, the pollens were filtered and air-dried overnight. Then they were transferred to orthophosphoric acid (400 ml) and refluxed for 7 days at 160° C. On the 8^{th} day, the pollens were separated from the acid by filtration and were washed with hot water (2×250 ml), acetone (250 ml), 2 mol/L hydrochloric acid (250 ml), 2 mol/L sodium hydroxide (250 ml), water (6×250 ml), acetone (250 ml), ethanol (2×250 ml). After overnight air drying, they were transferred to 6% KOH solution (800 ml). They were stirred under reflux for 12 hours at 120° C. with the solution renewed at 6 hours. After alkali reflux, the pollens were washed with hot water (6×250 ml), acetone (250 ml) and hot ethanol (2×250 ml) and then dried till constant weight in a hot air oven at 60° C.

To study the effect of temperature on the end product (treated pollen grains), the inventors obtained the above-mentioned treatment schemes were also performed at lower temperatures were reflux was not needed. In these schemes, the KOH treatment was carried out at 80° C. and the orthophosphoric acid treatment was carried out at 60° C. The acetone treatment was performed under reflux conditions as before.

Scanning electron microscopy. SEM analysis of different samples of pollens was performed using a field emission 54300 microscope from HITACHI (Japan). The samples were placed on a stainless steel stub with carbon tape and coated with gold and platinum using a Technics Hummer V Sputter Coater from Anatech USA (CA, USA) to enable visualization. Samples were imaged at different magnifications at an accelerating voltage of 2 kV.

Elemental analysis. Dried pollens (treated and natural) were analyzed using a calibrated PerkinElmer 2400 Series II CHNS/O analyzer. Next, 2 mg of dried pollens were used and all measurements were performed in triplicate. Percent nitrogen values obtained in this analysis were used to determine final protein concentration as follows:

Percent protein=Percent Nitrogen×6.25

where, 6.25 is the Kjeldahl conversion factor. [33]

TABLE 1
Abbreviations of the different treatment methods used in the study.

| Treatment name | Temperature | Abbreviation used |
|---|---|---|
| Conventional treatment | — | CT (aka CCT) |
| (aka Conventional | | |
| chemical treatment) | | |
| Modified conventional | — | MCT (aka MCCT) |
| treatment (aka Modified | | |
| conventional chemical | | |
| treatment) | | |
| Switched treatment | High | SCTH |
| (ST, aka Switched | Low | SCTL |
| chemical treatment) | | |

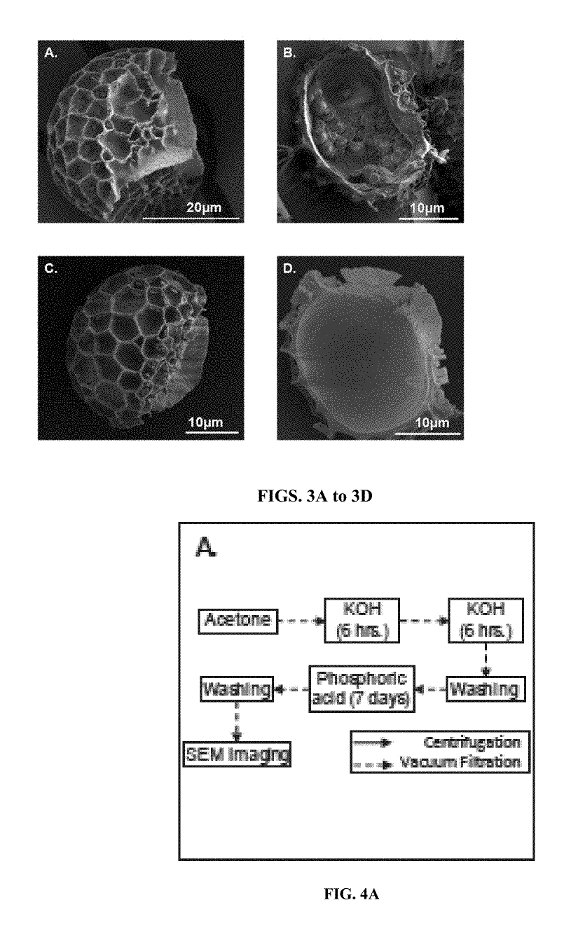

Conventional treatment (CT). As mentioned before, the method most commonly used for treatment of pollens involves sequential treatment of PGs with acetone, KOH and phosphoric acid. This treatment, known as the conventional treatment (CT) here, has been successfully used for obtaining clean Lycopodium spores (LSs) in published literature. The inventors were able to successfully obtain LSs that were morphologically intact with a clean surface and interior by this process. (FIGS. 3A to 3D).

FIGS. 3A to 3D show SEM images of Lycopodium spores (LSs) processed using the conventional treatment (CT). Raw LS: FIG. 3A shows the exterior showing the original morphology and FIG. 3B shows the interior showing the presence of natural biological material. LS processed using CT: FIG. 3C shows the exterior showing an intact morphology and FIG. 3D shows a clean interior made using the present invention.

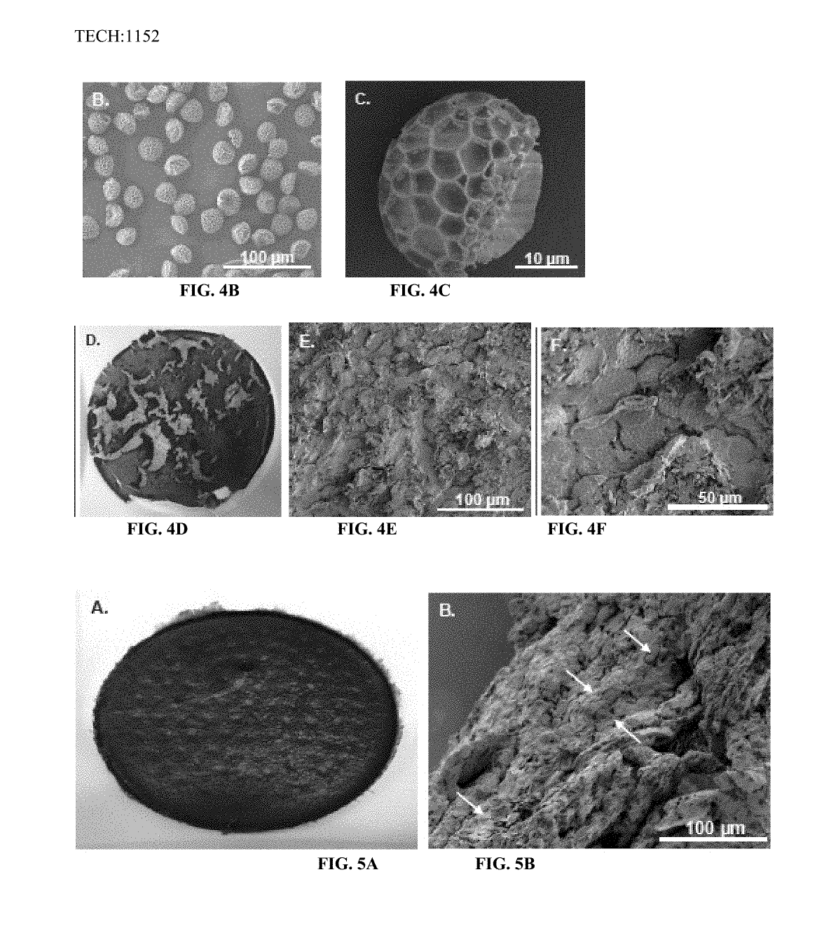

Similar to LSs, RW pollens were treated using CT. It was found that the pollens survive the acetone treatment with no visible damage. However, after 6 hours of KOH reflux and vacuum filtration, the pollens were found to form a thick layer (flake) on the filter paper from which the pollens could not be recovered. (FIG. 4A) This finding is in line with previous work, where the alkali treatment step has been reported to have damaging effect on the integrity of the pollen structure.^{[12, 26, 34]} However, the SEM image of this flake shows that the pollens may not be damaged as reported in previous literature but are in fact entrapped in some extraneous material. This causes them to stick together and become irrecoverable. (FIGS. 4B and 4C).

FIGS. 4A to 4G show a schematic diagram and images of LSs and RW pollens processed using the CCT and results therefrom. FIG. 4A is a schematic diagram of the processing steps on the CCT protocol. FIG. 4B shows LSs pollens after processing with CCT and FIG. 4C is a zoomed-in image of a single pollen. RW pollens after 6 hours of KOH treatment: FIG. 4D is a photograph of the flake formed after vacuum filtration. FIG. 4E is an SEM image of the flake showing pollen entrapped in extraneous materials. FIG. 4F is a zoomed-in SEM image of the flake showing more details of entrapped pollens.

Modified conventional treatment (MCT) for RW. To overcome the problem of irrecoverable pollens after KOH step and proceed to acid treatment, the inventors replaced the CT protocol. After acetone treatment, the pollens were separated by filtration and air-dried. Then they were refluxed for 12 hours in KOH with the solution renewed after 6 hours. During the alkali reflux the pollens were separated from solution by centrifugation to avoid loss due to filtration. After completion of the KOH treatment, these pollens were washed using hot water and ethanol using centrifugation where the washing solution (supernatant) was discarded at each step. At the final washing step, the pollens were separated from the solvent by vacuum filtration and air-dried. However, a similar flake formation was seen post this treatment.

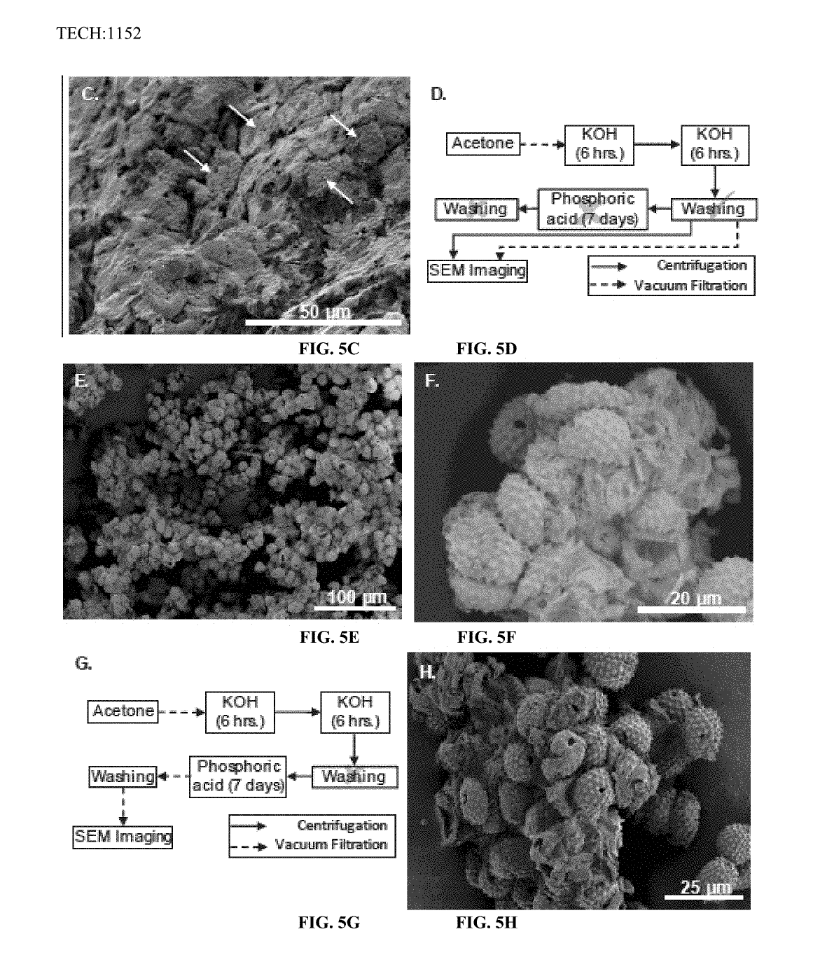

FIGS. 5A to 5I show images of RW pollens processed using the CCT and MCCT after 12 hours of KOH and MCCT after 7 days of phosphoric acid treatment. FIG. 5A shows a photograph of the flake formed after vacuum filtration. FIG. 5B. SEM image of the flake showing pollen entrapped in extraneous materials. FIG. 5C. Zoomed in SEM image of the flake showing more details of entrapped pollens. FIG. 5D. Schematic diagram of the processing steps for figures FIG. 5A to FIG. 5B (vacuum filtration) and FIG. 5E to FIG. 5F (centrifugation). FIG. 5E. Clumps formed after centrifugation and FIG. 5F. zoomed in SEM image of the clumps showing more details of entrapped pollens. FIG. 5G. Schematic diagram of the processing steps for figures FIG. 5H and FIG. 5I. FIG. 5H. SEM image of pollens clumped together and entrapped due to extraneous materials. FIG. 5I. Zoomed in SEM image of the clump showing more details of entrapped pollens with unclean surfaces.

To avoid this issue and proceed to the acid treatment step, in a separate set of experiments, after KOH reflux the pollens were transferred directly to 85% ortho-phosphoric acid without any washing steps in between. However, the pollens were found to form clumps in the acid after 24 hours (data not shown). The reflux was continued for 7 days and upon completion pollens were separated from acid by vacuum filtration and washed repeatedly with different solvents. The clumps formed in the early days of acid reflux were found to be retained. SEM images of these clumps reveal pollen surfaces which are dirty and sticking to each other.

The extraneous materials attaching to pollen surface are the natural biomolecules and organelles contained within the PGs that are released in to the surrounding solution as a result of the KOH treatment. By way of explanation, and in no way a limitation of the preset invention, the inventors hypothesize that this material that is released from pollens is in excess than the amount that can solubilize in the surrounding KOH solution. Hence, when PGs are separated from the alkali by vacuum filtration/centrifugation after the first 6 hours, some amount gets filtered with the aqueous phase while the remaining is stuck on the pollen surface causing them to form a flake. Centrifugation at this stage partially solves the problem making it possible to transfer pollens to fresh KOH solution for next 6 hours. However, further treatment in fresh KOH solution causes release of even more biological material, which further covers pollens and entraps them. At this point the entrapment is to a much greater extent and hence no matter what separation method is used, the pollens form aggregates (flake/clumps). Again, this issue can be partially resolved by removing the washing steps after KOH treatment and directly transferring the pollens to phosphoric acid. However, once in the acid the pollens were found to clump within 24 hours. This indicates that during KOH treatment the pollens get extensively entrapped in the released biological material and form aggregates (flake/clumps). These aggregates once formed cannot be broken by repeated washing or prolonged acid hydrolysis.

Switched treatment (ST) for RW. Based upon the above results it becomes clear that CT cannot yield intact and clean RW pollens. Hence, a new protocol was developed where the sequence of alkali and acid steps was switched. Briefly, post acetone reflux, the pollens were treated with ortho-phosphoric acid for 7 days. The recovered pollens were washed sequentially with different solvents. These were the further treated with KOH for 12 hours with the solution renewed at 6 hours. At each step the pollens were separated from the solvent by vacuum filtration. SEM images of these pollens show that they are morphologically intact with minimum damage. Next, RW pollens were processed using the ST protocol under non-reflux conditions (low temperatures) for the KOH and phosphoric acid step (STL). This was to determine whether reduced temperatures would yield a similar product and thereby make the process less harsh for the pollens.

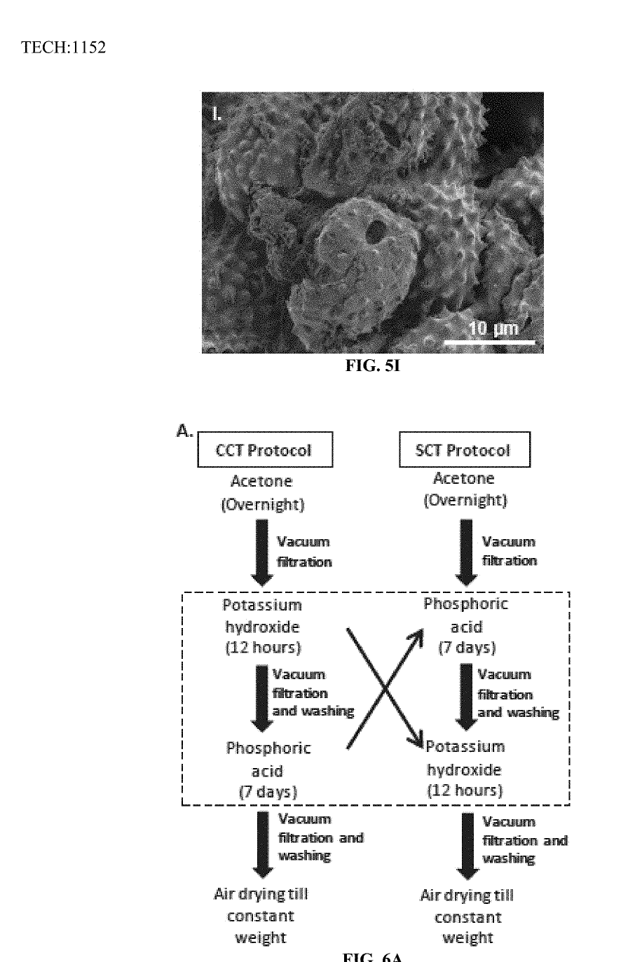

FIGS. 6A to 6J show SEM images of Ragweed (RW) pollens processed using the switched treatment (SCT). FIG. 6A shows a comparison diagram of the CCT and SCT treatment steps. Raw RW pollens: FIG. 6B is a zoomed-out image of multiple raw RW pollens, FIG. 6C shows an image of the exterior of the pollen showing the original morphology and FIG. 6D is a image that shows the interior of the pollen showing the presence of natural biological materials. RW pollens processed at high temperatures (SCTH): FIG. 6E is a zoomed-out image of multiple pollens after SCTH, FIG. 6F is an image of the exterior of a pollen showing an intact morphology and FIG. 6G is an image showing a clean interior of the processed pollen. RW pollens were processed at low temperatures (SCTL): FIG. 6H is a zoomed-out image of multiple pollen after SCTL, FIG. 6I is an image of the exterior of the pollen showing an intact morphology and FIG. 6J is am image showing a clean interior of the pollen.

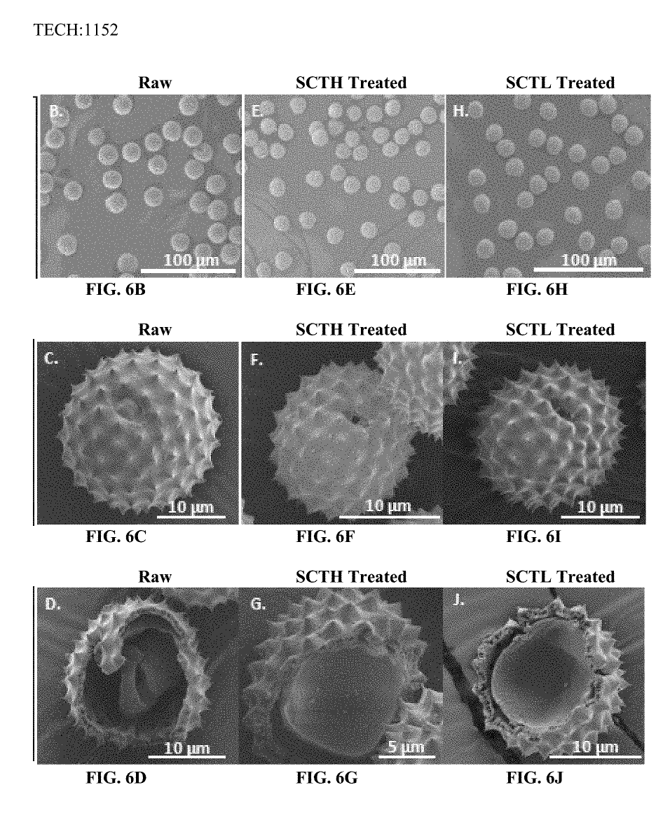

FIG. 7 is a graph that shows protein content of hollow exine shells obtained using the switched protocol A. The percent protein content of raw pollens and the ones processed by SCTH and SCTL show a considerable reduction indicating success of the process in removal of native proteinaceous material.

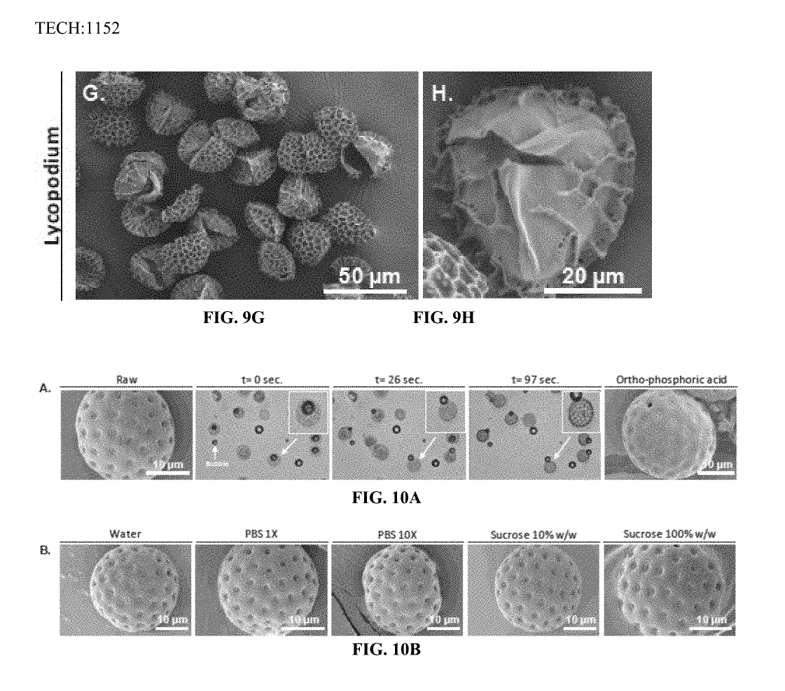

Based on these results, it can be said that the switching the sequence of steps, with acid treatment first followed by alkali treatment, the RW pollens were able to survive the entire process. It has been reported earlier that acid treatment is responsible for the maximum removal of natural biomolecules held within pollens.^{[11]} By way of explanation and in no way a limitation of the present invention, it was hypothesized that when defatted pollens are treated with phosphoric acid for a week, a large amount of biological material is released from the pollens. This biological material gets solubilized in the phosphoric acid and is removed during filtration. Thus after the phosphoric acid step, pollens are relatively empty. Hence, when next subjected to KOH treatment the amount of material released is much lower and can get solubilized in the surrounding KOH. This results in clean and intact RW pollens even at low temperatures. The percent protein content achieved with both the STH and STL protocols show that the method is successful in removing more than 90% of native biomolecules. (FIG. 10) The ST protocol was successful in obtaining clean intact SECs with other species of pollens.

Switched treatment (ST) for LSs. In order to determine whether the ST protocol can be used to replace the existing LSs treatment, LSs were treated using the STH protocol. It was interesting to note that LSs were unable to survive this process. The majority of defatted LSs seemed to burst open/crack at the trilete scar after the phosphoric acid step. Moreover, they were also seen to lose their surface morphology due to the treatment. To determine whether lower temperatures can reduce these adverse effects and give clean intact LSs, STL protocol was also tested. Similar results were seen with majority of pollens broken in the trilete scar area indicating that ST is not a suitable treatment protocol for LSs.

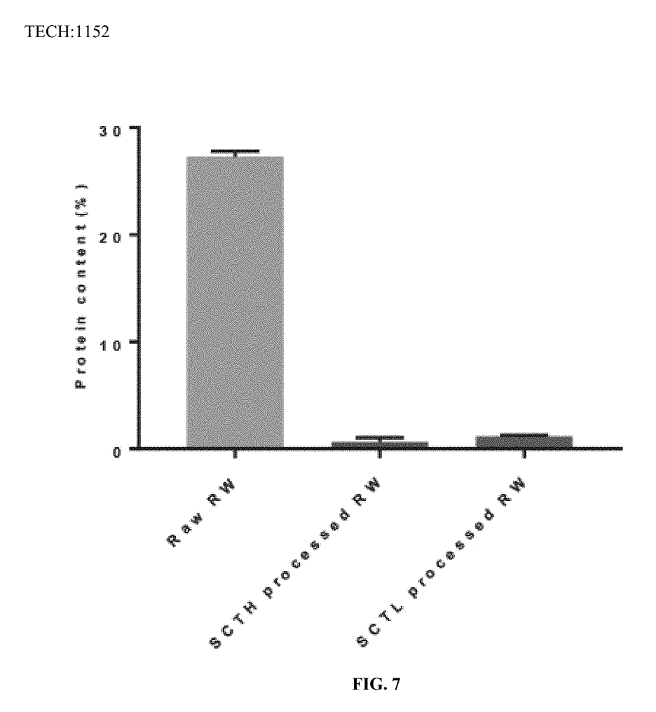

FIG. 8 shows a Fourier-transform infrared spectroscopy (FTIR) spectra of SCT processed RW pollen. Natural ragweed pollens were treated with acetone, phosphoric acid, and potassium hydroxide sequentially at two different temperatures. Low-temperature method used phosphoric acid and potassium hydroxide treatment at 60° C. and 80° C., respectively while high-temperature method used phosphoric acid and potassium hydroxide treatment at 160° C. and 120° C., respectively.

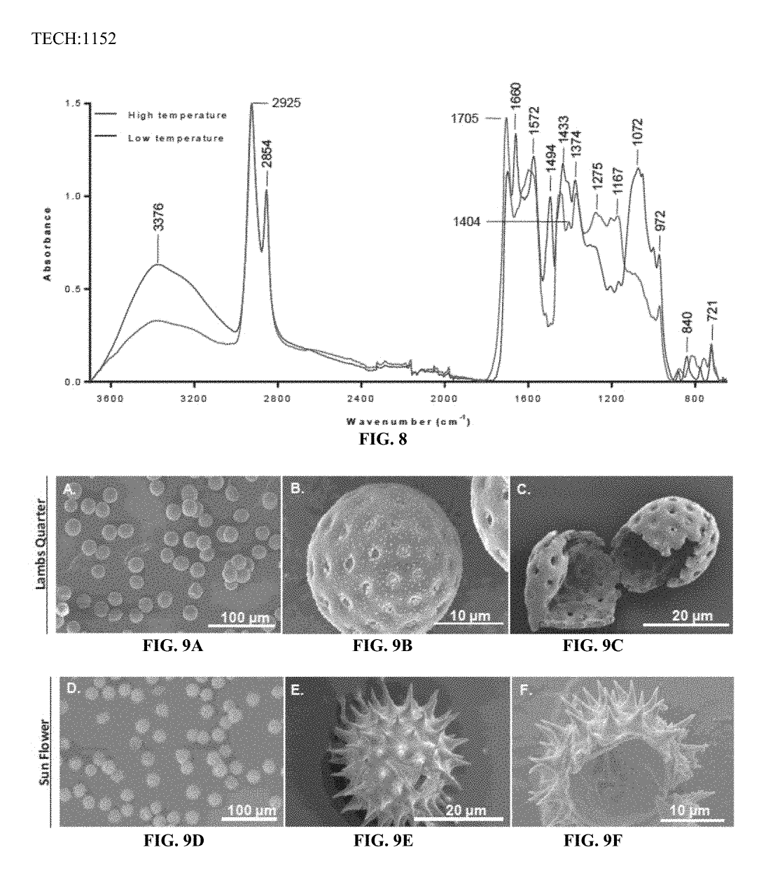

FIGS. 9A to H show SEM images of other species of pollens processed using the SCTL protocol. FIG. 9A. Zoomed out image of Chenopodium album (Lambs quarter), FIG. 9B. Intact processed pollen grain and FIG. 9C. clean interior achieved with the SCTL protocol. FIG. 9D. Zoomed out image of Helianthus annus (Sunflower) pollens, FIG. 9E. Intact processed pollen grain and FIG. 9F. clean interior achieved using the SCTL protocol. FIG. 9G. Zoomed out image of Lycopodium clavatum pollens and FIG. 9H. broken processed pollen grain as a result of the SCTL protocol.

Obtaining intact pollen shells of species other than Lycopodium clavatum has always been a challenge. Different methods do exist, but there is a need for a robust and optimized process that can be used with a variety of pollen species and results in clean and intact pollen shells with a low total protein content. In this study, the inventors investigated the cause of failure of the conventional treatment method, which is successful with LSs, for Ambrosia elation (Common ragweed) pollen. The results herein reveal that the alkali hydrolysis step results in entrapment of pollens from which they are irrecoverable. Even several modifications to the conventional treatment were unable to solve the problem. Hence, a new method was developed where the sequence of alkali and acid treatment steps was switched. This method was successful in producing clean and intact pollen shells of not only Ragweed but several other species of pollens. Even processing at low temperatures resulted in producing intact and clean pollens shells. The low temperature processing however results in a higher total protein content than that achieved using high temperatures. The switched method was found to be unsuccessful with LSs. When used, it resulted in considerable damage to the LSs with the pollens rupturing at the trilete scar. Based on these results the inventors conclude that; the switched protocol can be applied to pollen species that have obvious apertures on their surface that facilitate release of dissolved biological material in the phosphoric acid step. This prevents rupture of the pollen due to osmotic shock as is seen in LSs. This finding is important as it provided a robust and provides a well-optimized protocol for processing multiple species of pollens to obtain clean intact shells that can be further used for various applications.

FIGS. 10A and 10B show images of pollen apertures bursting due to osmotic pressure buildup. FIG. 10A shows Lambs Quarter (LQ) pollens before and after exposure to ortho-phosphoric acid showing the buildup of pressure that will cause the opening to burst open to release it. FIG. 10B. LQ pollens SEM images after exposure to other solvents that did not cause a buildup in pressure.

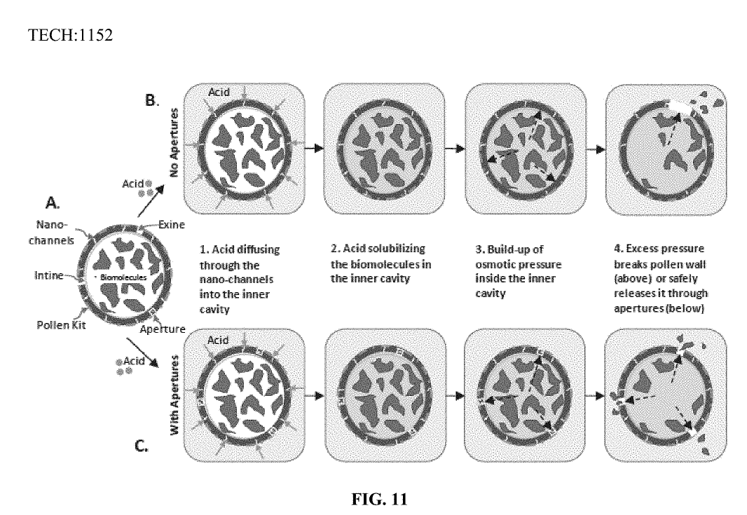

By way of explanation, and in no way a limitation of the present invention, FIG. 11 shows a proposed mechanism of pore opening in pollen grains. At point A, a diagram of a pollen grain with its different components is shown. In pathway B, a diagram of pollen without aperture exposed to an environment that causes a build up in osmotic pressure, which release will be at a weak spot on the pollen wall. In pathway C, a diagram of pollen with an aperture where the buildup pressure will be release through the pores.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Atkin, S. L., S. T. Beckett, and G. Mackenzie, Dosage form, 2006, Google Patents.
2. Amer, M. S. and R. Tawashi, Modified pollen grains for delivering biologically active substances to plants and animals, 1991, Google Patents.
3. Amer, M. S. and R. Tawashi, Drug loaded pollen grains with an outer coating for pulsed delivery, 1994, Google Patents.
4. Erdtman, G., The acetolysis method. A revised description. Svensk Bot. Tidsk, 1960. 54: p. 561-4.
5. Dominguez, E., et al., Isolation of intact pollen exine using anhydrous hydrogen fluoride. Grana, 1998. 37(2): p. 93-96.
6. Baldi, B., V. Franceschi, and F. Loewus, Preparation and properties of pollen sporoplasts. Protoplasma, 1987. 141(1): p. 47-55.
7. Ahlers, F., J. Lambert, and R. Wiermann, Structural elements of sporopollenin from the pollen of Torreya californica Torr. (Gymnospermae): Using the 1H-NMR technique. Zeitschrift für Naturforschung C, 1999. 54(7-8): p. 492-495.
8. Gubatz, S., et al., Tracer experiments on sporopollenin biosynthesis: an overview. Grana, 1993. 32(S1): p. 12-17.
9. Mackenzie, G., et al., Chapter 24—Pollen and Spore Shells—Nature's Microcapsules, in Microencapsulation in the Food Industry 2014, Academic Press: San Diego. p. 283-297.
10. Diego-Taboada, A., et al., Hollow pollen shells to enhance drug delivery. Pharmaceutics, 2014. 6(1): p. 80-96.
11. Mundargi, R. C., et al., Eco-friendly streamlined process for sporopollenin exine capsule extraction. Sci Rep, 2016. 6(19960).
12. Mundargi, R. C., et al., Extraction of sporopollenin exine capsules from sunflower pollen grains. RSC Advances, 2016. 6(20): p. 16533-16539.
13. Human, H. and S. W. Nicolson, Digestion of maize and sunflower pollen by the spotted maize beetle Astylus atromaculatus (Melyridae): is there a role for osmotic shock? Journal of Insect Physiology, 2003. 49(7): p. 633-643.
14. Bohne, G., H. Woehlecke, and R. Ehwald, Water relations of the pine exine.

Annals of Botany, 2005. 96(2): p. 201-208.
15. Matamoro-Vidal, A., et al., Links between morphology and function of the pollen wall: an experimental approach. Botanical Journal of the Linnean Society, 2016.
16. Bedinger, P., The remarkable biology of pollen. Plant Cell, 1992. 4(8): p. 879-87.
17. Borg, M., L. Brownfield, and D. Twell, Male gametophyte development: a molecular perspective. J Exp Bot, 2009. 60(5): p. 1465-78.
18. Mascarenhas, J. P., The Male Gametophyte of Flowering Plants. The Plant Cell, 1989. 1(7): p. 657-664.
19. Katifori, E., et al., Foldable structures and the natural design of pollen grains. Proceedings of the National Academy of Sciences, 2010. 107(17): p. 7635-7639.
20. Edlund, A. F., R. Swanson, and D. Preuss, Pollen and Stigma Structure and Function: The Role of Diversity in Pollination. The Plant Cell, 2004. 16(suppl 1): p. S84-S97.
21. Shivanna, K. R., Pollen Biology and Biotechnology 2003: Science Publishers.
22. Roulston, T. H. and J. H. Cane, Pollen nutritional content and digestibility for animals. Plant Systematics and Evolution, 2000. 222(1): p. 187-209.
23. BOHNE, G., et al., Diffusion Barriers of Tripartite Sporopollenin Microcapsules Prepared from Pine Pollen. Annals of Botany, 2003. 92(2): p. 289-297.
24. Ariizumi, T. and K. Toriyama, Genetic Regulation of Sporopollenin Synthesis and Pollen Exine Development. Annual Review of Plant Biology, 2011. 62(1): p. 437-460.
25. Park, J. H., et al., Inflated Sporopollenin Exine Capsules Obtained from Thin-Walled Pollen. Scientific Reports, 2016. 6: p. 28017.
26. Barrier, S., Physical and chemical properties of sporopollenin exine particles, 2008, University of Hull.
27. Atwe, S. U., Y. Ma, and H. S. Gill, Pollen grains for oral vaccination. Journal of Controlled Release, 2014. 194(0): p. 45-52.
28. Diego-Taboada, A., et al., Protein free microcapsules obtained from plant spores as a model for drug delivery: ibuprofen encapsulation, release and taste masking. Journal of Materials Chemistry B, 2013. 1(5): p. 707-713.
29. Lorch, M., et al., Mill contrast agent delivery using spore capsules: controlled release in blood plasma. Chem Commun, 2009. 14(42): p. 6442-4.
30. Hamad, S. A., et al., Encapsulation of living cells into sporopollenin microcapsules. Journal of Materials Chemistry, 2011. 21(44): p. 18018-18023.
31. Wakil, A., et al., Enhanced bioavailability of eicosapentaenoic acid from fish oil after encapsulation within plant spore exines as microcapsules. Lipids, 2010. 45(7): p. 645-9.
32. Barrier, S., et al., Sporopollenin exines: A novel natural taste masking material. LWT—Food Science and Technology, 2010. 43(1): p. 73-76.
33. Beckett, S. T., S. L. Atkin, and G. Mackenzie, Dosage form, 2009, Google Patents.
34. Southworth, D., Solubility of Pollen Exines. American Journal of Botany, 1974. 61(1): p. 36-44.

What is claimed is:

1. A method of making a hollow exine shell from pollen grains comprising the steps of:
providing a plant pollen or spore;
extracting organic matter from the plant pollen or spore with an organic solvent;
after the organic extraction treating the plant pollen or spore with an acid solution;
after the acid treatment treating the plant pollen or spore with an alkali solution; and
isolating the plant pollen or spore, wherein the pollen or spore have open apertures on pollens with visible apertures that open to the interior hollow cavity, wherein the same apertures are closed in naturally occurring pollens.

2. The method of claim 1, further comprising the step of changing the times for at least one of the organic extraction, acid treatment, or the alkali treatment to optimize the size of the apertures.

3. The method of claim 1, further comprising the step of changing the strength of the acid to optimize the size of the aperture of the plant pollen or spore.

4. The method of claim 1, further comprising the step of changing the strength of the alkali to optimize the size of the aperture of the plant pollen or spore.

5. The method of claim 1, further comprising the step of adding an antigen selected from peptides, proteins, bacteria, viruses, fungi, protozoans, parasites, prions, toxins, cancer, or allergens including food allergens to modulate an immune response to the antigen.

6. The method of claim 1, further comprising the step of adding one or more antigens comprising oligonucleotides, proteins, peptides, deoxyribonucleic acid (DNA), ribonucleic acid (RNA), cells (broken or intact), lipids, toxin variants, carbohydrates, virus-like particles, liposomes, live attenuated or killed natural or recombinant microorganisms, bacteria, viruses, and particulate vaccine delivery systems, liposomes, virosomes, polymeric/inorganic/organic micro and nanoparticles, immune stimulating complexes (ISCOMS) and combinations thereof, wherein antigens are in composition or can be attached/adsorbed/anchored physically or chemically to pollen/spore at the exterior surface, interior surface/cavity or pores.

7. The method of claim 1, wherein the plant pollen or spore is formed into a vaccine composition that is adapted for oral, nasal, pulmonary, rectal, occular, transdermal, transmucosal, intramuscular, or subcutaneous delivery.

8. The method of claim 1, further comprising the step of coating the treated plant pollen or spore with a coating.

9. The method of claim 1, further comprising the step of adding at least one of an adjuvant or an antigenic protein to the treated plant pollen or spore.

10. The method of claim 1, wherein the isolated pollen is at least one of: substantially free of proteins, substantially free to antigenic proteins, free of proteins, or free of antigenic proteins.

11. The method of claim 1, wherein each of the steps of extracting, or treating are followed by a vacuum filtration and washing step.

12. The method of claim 1, further comprising the step of adding a polymer coating applied to the pollen/spore, wherein the polymer coating is a diffusion barrier, a coating that includes physical or chemical adsorption/attachment/anchoring points, plugs one or more of the multiple pores, coats the inner cavity, coats the exterior surface or a combination thereof.

13. An open pore plant pollen or spore made by a method that comprises the steps of:
providing a plant pollen or spore;
extracting organic matter from the plant pollen or spore with an organic solvent;
after the organic extraction treating the plant pollen or spore with a hot strong acid solution;
after the acid treatment treating the plant pollen or spore with a hot strong alkali solution; and
isolating the plant pollen or spore, wherein the pollen or spore have open apertures on pollens with visible apertures that open to the interior hollow cavity, wherein the same apertures are closed in naturally occurring pollens.

14. The method of claim 14, further comprising the step of changing the times for at least one of the organic extraction, acid treatment, or the alkali treatment to optimize the size of the apertures.

15. The method of claim 14, further comprising the step of changing the strength of the acid to optimize the size of the aperture of the plant pollen or spore.

16. The method of claim 14, further comprising the step of changing the strength of the alkali to optimize the size of the aperture of the plant pollen or spore.

17. The method of claim 14, further comprising the step of adding an antigen selected from peptides, proteins, bacteria, viruses, fungi, protozoans, parasites, prions, toxins, cancer, or allergens including food allergens to modulate an immune response to the antigen.

18. The method of claim 14, further comprising the step of adding one or more antigens comprising oligonucleotides, proteins, peptides, deoxyribonucleic acid (DNA), ribonucleic acid (RNA), cells (broken or intact), lipids, toxin variants, carbohydrates, virus-like particles, liposomes, live attenuated or killed natural or recombinant microorganisms, bacteria, viruses, and particulate vaccine delivery systems, liposomes, virosomes, polymeric/inorganic/organic micro and nanoparticles, immune stimulating complexes (ISCOMS) and combinations thereof, wherein antigens are in composition or can be attached/adsorbed/anchored physically or chemically to pollen/spore at the exterior surface, interior surface/cavity or pores.

19. The method of claim 14, wherein the plant pollen or spore is formed into a vaccine composition that is adapted for oral, nasal, pulmonary, rectal, occular, transdermal, transmucosal, intramuscular, or subcutaneous delivery.

20. The method of claim 14, wherein the vaccine composition is a liquid, a solid, an aerosolized or a combination thereof.

21. The method of claim 14, further comprising the step of adding at least one of an adjuvant or an antigenic protein to the treated plant pollen or spore.

22. The method of claim 14, further comprising adding a polymer coating applied to the pollen/spore, wherein the polymer coating is a diffusion barrier, a coating that includes physical or chemical adsorption/attachment/anchoring points, plugs one or more of the multiple pores, coats the inner cavity, coats the exterior surface or a combination thereof.

23. The method of claim 14, wherein the strong acid is selected from sulfuric acid, nitric acid, phosphoric acid, hydrobromic acid, hydroiodic acid, chloric acid, and hydrochloric acid.

24. The method of claim 14, wherein the strong base is selected from sodium hydroxide, potassium hydroxide, lithium hydroxide, calcium hydroxide, or barium hydroxide.

25. The method of claim 14, wherein the organic solvent is selected from acetone, methyl acetate, ethyl acetate, acetonitrile, dimethylformamide, tetrachloroethylene, toluene, 1,4-dioxane, chloroform, diethyl ether, dichloromethane, turpentine, pentane, hexane, cyclohexane, benzene, ethers, or citrus terpenes.

26. The method of claim 14, further comprising the step of coating the treated plant pollen or spore with a coating.

27. The method of claim 14, further comprising the step of adding at least one of an adjuvant or an antigenic protein to the treated plant pollen or spore.

28. The method of claim 14, wherein the isolated pollen is at least one of: substantially free of proteins, substantially free to antigenic proteins, free of proteins, or free of antigenic proteins.

29. The method of claim 14, further comprising the step of adding a polymer coating applied to the pollen/spore, wherein the polymer coating is a diffusion barrier, a coating that includes physical or chemical adsorption/attachment/anchoring points, plugs one or more of the multiple pores, coats the inner cavity, coats the exterior surface or a combination thereof.
